(19) United States
(12) Patent Application Publication
(10) Pub. No.: US 2009/0215079 A1
(43) Pub. Date: Aug. 27, 2009

(54) Method and Devices for the Detection of Microorganisms and/or the Activity Thereof

(75) Inventors: Kai Ostermann, Dresden (DE); Wolfgang Pompe, Hartha (DE); Dagmar Wersing, Dresden (DE); Michael Mertig, Dresden (DE); Justin Gooding, Sydney (AU); Gerhard Rödel, Karlsfeld (DE); Karolina Ihle, Dresden (DE)

Correspondence Address: GUDRUN E. HUCKETT DRAUDT, SCHUBERTSTR. 15A, WUPPERTAL, 42289, DE

(73) Assignee: TECHNISCHE UNIVERSITaT DRESDEN, Dresden (DE)

(21) Appl. No.: 12/096,073

(22) PCT Filed: Dec. 1, 2006
(86) PCT No.: PCT/DE2006/002190

§ 371 Date: Oct. 27, 2008

(30) Foreign Application Priority Data
Dec. 5, 2005 (DE) 10 2005 059 483.2

(51) Int. Cl. G01N 33/53; C12M 1/00; G01N 27/26

(52) U.S. Cl. 435/7.2; 435/287.1; 435/288.7; 204/403.01

(57) ABSTRACT

In a method for detection of microorganisms and/or their activity with biosensors, on a surface of a substrate over portions thereof at least one ligand for binding a receptor or at least one receptor for a ligand or at least one ligand for binding a receptor and at least one receptor for either the one or a further ligand are immobilized chemically, physically, or biologically, wherein physical or physicochemical changes, caused at receptors by binding of ligands that are emitted by microorganisms in the process of quorum sensing are measured.

The invention concerns a method for detection of microorganisms and/or their momentary state of activity by means of biosensors that are based on quorum sensing-components as well as devices functioning as biosensors for performing the method to be used, for example, in biotechnology and environmental technology as well as medical technology.

Bacterial contaminations in drinking water or in biotechnological production processes, for example, in yeast fermentation or in cell cultures, present a serious and often health-hazardous problem. In order to introduce suitable countermeasures it is necessary to detect the bacteria as early as possible.

The control of activity of microorganisms in biochemical processes for obtaining optimal yields requires methods for detecting the momentary state of cells.

Microbiological analyses, i.e., taking samples and assaying the germs by culturing on selective culture media, have a series of disadvantages: on the one hand taking the sample presents a possible source of contamination; on the other hand, culturing and analysis of the bacteria is time-intensive and cost intensive. Moreover, the microbiological analysis allows detection of biofilm-forming bacteria only to a very limited extent. By methods of molecular biology for example, PCR analyses, the detection time can be shortened, but the necessity of taking a sample and isolating the DNA makes this method also rather slow. By selection of the primers for the PCR reactions or probes in case of hybridization, one accepts a great limitation of the spectrum of bacteria that can be detected. Moreover, with this method no differentiation between living and dead microorganisms is possible.

Object of the invention is to provide methods and means for detection of microorganisms and/or their activity that enable the fast and early detection of microorganisms.

According to the invention, this object is solved by a method for detection of microorganisms and/or their activity with biosensors wherein on the surface of a substrate, over portions thereof, at least one ligand for binding a receptor or at least one receptor for a ligand or at least one ligand for binding a receptor and at least one receptor for either the one or a further ligand are immobilized chemically, physically, or biologically, wherein physical or physical-chemical changes, caused at the receptors by binding of ligands that are emitted by microorganisms in the process of quorum sensing, can be measured.

The object is also solved by a device for detection of microorganisms and/or their activity as biosensor, wherein on the surface of a substrate, over portions thereof, at least one ligand for binding a receptor or at least one receptor for a ligand or at least one ligand for binding a receptor and at least one receptor for the one or the further ligand are immobilized chemically, physically, or biologically, wherein physical or physical-chemical changes, caused by bonds between ligands, emitted by microorganisms in the process of quorum sensing, and the receptors, are measured.

The methods and devices are characterized in particular in that a fast and early detection of a greater bandwidth as well as a certain type of bacteria species and/or its activity is possible.

For this purpose, on the surface of a substrate, over portions thereof, at least one ligand for binding a receptor or at least one receptor for a ligand or at least one ligand for binding a receptor and at least one receptor for either the one or a further ligand are immobilized chemically, physically or biologically, wherein physical or physical-chemical changes, caused at the receptors by binding of ligands that have been emitted by microorganisms in the process of quorum sensing, can be measured.

For this purpose, on the surface of a substrate, over portions thereof, at least one ligand for binding a receptor or at least one receptor for a ligand or at least one ligand for binding a receptor and at least one receptor for either the one or a further ligand are immobilized chemically, physically or biologically, wherein physical or physical-hemical changes, caused by bonds between ligands emitted by microorganisms in the process of quorum sensing and the receptors, are measured.

The principle of the invention is to utilize the communication between microorganisms for physical or physical-technical processes.

Quorum sensing is a term for the communication between microorganisms by means of small hormone-like signaling molecules, so-called autoinducers, as ligands and receptors. This process plays a decisive role in the regulation of cell density enabling bacteria populations to behave similar to multi-cell organisms and to benefit from advantages in this way. The behavior patterns that are regulated by quorum sensing include, inter alia, the production of antibiotics, symbiosis, conjugation, virulence, formation of biofilms as well as bioluminescence of some Vibrio species.

The signaling molecules referred to as autoinducers are produced by certain bacteria with the aid of certain genes and released into the surrounding culture medium. Bacteria have a matching receptor system for these ligands. After reaching a certain concentration of signaling molecules in the medium, the autoinducer signaling molecules will bind to the receptor. The receptor-autoinducer complex transmits the information to the interior of the cell and activates in this way transcription of certain genes.

There are different classes of autoinducers. Usually they are species-specific, i.e., different bacteria species produce different signaling molecules for communication among their own bacteria species. The autoinducer-2 (in the following also referred to as AI-2) enables, for example, communication between different bacteria species (interspecies communication) and therefore represents a universal signaling molecule in the communication between microorganisms.

More than 30 different mostly gram-negative bacteria types, including Escherichia coli, Salmonella typhimurium, Salmonella paratyphi, Helicobacter pylori, Vibrio cholerea, Shigella flexneri, Staphylococcus aureus, produce the signaling molecule autoinducer-2 with the aid of a certain gene (LuxS or homologs), secrete it into the culture medium, and have a matching intra-cellular receptor system (Schauder, S., Shokat, K., Surette, M. G., and Bassier, B. L. (2001); The LuxS-family of bacterial autoinducers: Biosynthesis of a novel quorum sensing signal molecule. Mol. Microbiol. 41: 463-476; Schauder S. and Bassler, B. L. (2001) The language of bacteria. Genes Dev. 15: 1468-1480; Sperandio, V., Torres, A. G., and Kaper, J. B. (2002) Quorum sensing Escherichia coli regulators B and C (QseBC): a novel two-component regulatory system involved in the regulation of flagella and motility by quorum sensing in Mol. Microbiol. 43: 809-821.)

This special form of quorum sensing enables the bacteria to communicate among different species. In contrast to conventional quorum sensing, the different bacteria species produce a same-type signal. Proteins of the LuxP type act as receptors. LuxP is present as a soluble protein in the periplasm and binds AI-2. The complex of LuxP and AI-2 transmits by a two-component system a signal into the interior of the cell.

According to the invention, the ligands that are secreted by the microorganisms in the process of quorum sensing are detected by physical or physical-chemical methods for detecting microorganisms.

For this purpose, the components (receptor or ligand) of the bacterial quorum sensing system are used for functionalizing surfaces of substrates. Such surfaces can also be simultaneously the surfaces of sensors so that significant advantageous configurations result. Quorum sensing is disclosed also for eukaryotes, for example, such as e.g. yeasts, wherein the basic mechanisms are partially unclear. But in this case a corresponding utilization of receptor and ligand of the quorum sensing system is possible also.

For functionalizing the surface of the substrate, signaling molecules such as species-specific autoinducer, for example, autoinducer-1 or autoinducer of the inter-species communication, for example, AI-2, as ligands and corresponding receptors are utilized. The signaling molecules can be, on the one hand, the authentic molecules released by the respective organism that are either directly produced by the microorganism or are expressed heterologously by a host organism or are chemically synthesized. However, any other type of biologically and/or chemically produced molecule can be used with which the corresponding quorum sensing receptor or a derivative of a quorum sensing receptor can interact.

Examples of specific autoinducers or their respective receptors are based in case of gram-negative bacteria on the Luxl/LuxR system (Swift, S, Karlyshew, A. V., Fish, L., Durant, E. L., Winson, M. K., Chhabra, S. R., Williams, P., Macintrye, S., and Stewart, G. S. A. B. (1997) Quorum Sensing in Aeromonas hydrophilia and Aeromonas salmonicidia: Identification of the LuxRI homologs AhyRI and AsaRI and their cognate N-acylhomoserine lactone signal molecules. J. Bacteriol. 179: 5271-5281). In this connection, LuxI refers to the autoinducer synthase and LuxR to the autoinducer receptor. The autoinducers-1 are generated from acylated homoserine lactones (AHL). For the production LuxI is decisive. The produced autoinducers differ from one another primarily based on the length of their acyl chain and/or their degree of saturation. The highly specific recognition of the respective autoinducer-1 is ensured by an appropriate receptor. Up to now, for more than 50 species corresponding quorum sensing systems have been identified (DeLisa, M. P. and Bentley, E. B. (2002) Bacterial autoinduction: looking outside the cell for new metabolic engineering targets, Microb. Cell Fact. 1:5)). Each one of the corresponding receptors or autoinducers-1 or corresponding derivatives of receptors or autoinducers is suitable for the method according to the invention.

In gram-positive bacteria, instead of AHL post-translationally processed peptide signaling molecules are used as autoinducer (de Kievit, T. R. and Iglewski, B. H. (2000) Bacterial quorum sensing in pathogenic relationships. Infect. Immun. 68: 4839-4849; Winans, S. C. and Bassler, B. L. (2002) Mob psychology. J. Bacteriol. 184: 873-883). A membrane-based histidine kinase of a two-component signal system acts as a receptor. Components of this system can also be used in accordance with the invention. As an example the signal transduction of Staphylococcos aureus should be mentioned (de Kievit, T. R. and lglewski, B. H. (2000) Bacterial quorum sensing in pathogenic relationships. Infect Immun. 68: 4839-4849; Winans, S. C. and Bassler, B. L. (2002) Mob psychology. J. Bacteriol. 184: 873-883). The signal peptide is processed from the precursor peptide of the agrD gene product. The membrane-based AgrC protein serves as a receptor.

When species-specific ligands or the corresponding receptors are utilized, the detection system is especially suitable e.g. for selective monitoring of certain pathogenic microorganisms and their activity in medical, pharmaceutical and food-chemical area.

Particularly preferred is the use of AI-2 or the use of LuxP receptors or its homologs as, for example, the LuxP receptor of Vibrio harveyi. By using the quorum sensing components of the inter-species communication of microorganisms, the simultaneous detection of many different bacteria species is possible.

The receptors of the quorum sensing systems that are used for functionalizing the sensor surface are preferably produced by recombinant protein synthesis in microorganisms such as, for example, Escherichia coli. In this connection, the corresponding genes of, for example, genomic DNA or c-DNA are PCR-amplified, produced as synthetic genes or appropriate fusion genes are produced by suitable recombinant DNA techniques and cloned in suitable expression vectors, for example, pET-23, novagen, and optionally provided with a protein tag for purification or optionally binding. After expression of the proteins, the purification of the proteins takes place, for example, by means of affinity chromatography by Ni-NTA columns (Qiagen). Alternatively, other expression systems or host organisms such as, for example, yeasts can be used.

For immobilization the receptor is bonded by a suitable linker to the substrate surface. The immobilization on the substrate surface is done such that the functionality of the receptors is not impaired. The binding of the receptor proteins is realized preferably by methods known to a person skilled in the art for binding proteins to substrate surfaces as summarized, for example, in Cao, L., Curr Opin Chem Biol 2005, April 9 (2):217-26

Suitable binding possibilities are provided, for example, by the following reaction pathways. For a surface of the substrate made from gold the linker is bonded by means of a thiol to the gold surface, for example, 11-mercapto undecane acid, and forms by way of a carboxylic acid group activated by N-(3-dimethylaminopropyl)-N′-ethylcarbodiimide (EDC) and N-hydroxy sulfosuccinimide (NHS) an amide bond with primary amines of the proteins. In the case of substrates with metal oxide surfaces the protein is bonded to the substrate surface by a silane that presents epoxide groups, for example, 3-glycidyloxypropyl trimethoxysilane, and forms with the primary amines of the protein secondary amine bonds.

The signaling molecules that are used for functionalizing the substrate surface are chemically synthesized, preferably according to Semmelhack, M. F., Campagna, S. R., Federle, M. J., Bassler, B. L. “An expeditious synthesis of DPD and boron binding studies”, Org. Lett. 2005 Feb. 17: 7(4): 569-72.

For immobilizing the chemically synthesized signaling molecules, the autoinducer molecule is bonded to the substrate, for example, by a homobifunctional cross-linker, for example, 1,4 butanediol diglycidyl ether, that forms ether bonds with the hydroxyl group of the autoinducer. Binding to the surface of the substrate is realized by the cross-linkers bonding to layers on the substrate that present amino groups; cysteine monolayer in case of gold surfaces or amino silanes or amino terpolymers in the case of oxide surfaces. The autoinducer molecule can also be bonded to the gold surface of the substrates by EDC/NHS activated 11-mercapto undecane acid.

In order to prevent non-specific binding of molecules to the substrate surface, an antifouling molecule, for example, hexaethylene glycol, is bonded to the substrate surface simultaneously with the protein or the autoinducer. The antifouling molecule blocks the surface areas that are not occupied by protein or autoinducer by means of its special free end groups in such a way that no other molecules can bind thereat.

Advantageous embodiments of the invention are provided in claims 2 to 19 and 21 to 27.

In the embodiment according to claim 2, to a surface of the substrate over portions thereof at least one ligand is bonded, receptors are bonded to ligands and receptors bind ligands that are released by the microorganisms in the process of quorum sensing and/or already present in the medium. In this connection, receptors are released with ligands of the medium. This release is measuring-technologically detected by the release-caused changes of charge, mass, optical properties, magnetic properties, conformation or enzymatic activity.

The ligand, in the embodiment of claim 3, is advantageously a signaling molecule.

On a surface of a substrate over portions thereof, according to the embodiment of claim 4, at least receptors are bonded and signaling molecules of the medium that have been released by the microorganisms in the process of quorum sensing and/or are already present in the medium are bonded to receptors, wherein a physicochemical change happens so that measuring-technologically the thus caused changes of the charge, the mass, the conformation, the optical properties, the magnetic properties, the charge distribution or the enzymatic activity are assayed.

In case of the change of optical properties dyes can be coupled.

The biosensor, according to the embodiment of claim 5, is advantageously calibrated by attaching particles, for example, particles of defined mass, to the ligands or receptors. Preferably, the particles are metal clusters of defined mass, in particular gold clusters. Calibration can also be realized by predetermined particle concentrations of same or different particle types, for example, by adding defined amounts of AI-2.

The surface of the substrate with signaling molecules and thus bonded receptors according to the embodiment of claim 2 is regenerated according to the embodiment of claim 6 advantageously by repeated coating with the receptor.

For increasing the sensitivity, according to the embodiment of claim 7, for generating fusion proteins by means of recombinant techniques amino acid sequences of the receptors are modified such that a change of charge, mass, light, conformation or enzymatic reaction happens and/or a change of charge, mass, light, conformation or enzymatic reaction is realized chemically or physically. In this way, fusion proteins are generated on the basis of quorum sensing receptors, whose properties are changed by means of recombinant techniques such that the signals detected by the respectively used sensor type in the form of charge, mass, conformation, light, magnetic field strength, enzymatic reaction are changed. The fusion concept leads to a measurable change of the signals to be detected specifically for the physical-chemical sensor, respectively.

According to the embodiment of claim 8, the receptors are changed by fusion with charged peptides to fusion proteins with changed net charge. This embodiment is especially suitable for sensor types that are based on charge change. The fusion is realized preferably at the DNA level by means of suitable recombinant DNA techniques. A charge change can also be realized chemically. According to the embodiment of claim 9 the peptides contain at positive net charge preferably arginine, histidine or lysine, and for a negative net charge aspartic acid or glutamic acid.

According to the embodiment of claim 10, the receptors are changed by fusion with proteins or peptides such that the mass of the fusion proteins is significantly increased. This embodiment is particularly suitable for sensor types that are based on assaying by mass changes. The fusion is realized, for example, with β-galactosidase of E. coli. The fusion is realized preferably at the DNA level by means of suitable recombinant DNA techniques. The mass change can also be realized differently, for example, chemically. For example, a mass change can be realized by biotinylation.

According to the embodiment of claim 11, the receptors are changed by fusion with proteins of peptides that have auto fluorescence or that cause a luminous reaction enzymatically by addition of suitable substrate molecules. This embodiment is particularly suitable for sensor types whose measuring principle is based on the detection of qualitative for quantitative changes of emission of incident light. The fusion is realized, for example, by proteins or peptides that have auto fluorescence, for example, green-fluorescent protein or its derivatives or that are able to cause enzymatically by addition of suitable substrate molecules a luminous reaction, for example, luciferase. Moreover, all enzyme reactions can be utilized that can be followed measuring technologically. The fusion is realized preferably at the DNA level by means of suitable recombinant DNA techniques. The fusions can also be done chemically.

While the aforementioned modifications are specific to the respective sensor type, a general signal amplification is achieved by the embodiment of claim 12 in that receptors are changed in their amino acid sequences such that the affinity between signaling molecule and receptor is increased or decreased. For this purpose, the amino acids that are important for binding of autoinducers are changed in a targeted fashion by suitable DNA recombinant techniques (for example, direct mutagenesis by means of PCR).

In this connection, the receptors can be changed in their amino acid sequences such that the binding affinity between signaling molecule and receptor for the respective sensor type is optimized. This can be done by modification of individual amino acids of the receptors that cause increased or decreased affinity of binding of the signaling molecule. Optionally, combinations of several modified amino acids can be used also.

It is also possible to make changes that affect the steric orientation of the receptors. In accordance with the embodiment of claim 13, for detection of a minimal number of signaling molecules that are emitted by microorganisms in the process of quorum sensing an additional protein is added to the receptors by means of recombinant techniques so that fusion proteins are generated. Either the fusion proteins or the signaling molecules are bonded to the surface of the substrate by amino acid or nucleic acid sequences or lipids that act sensitively relative to the enzymatic activity of the fusion proteins; in this way, the release of a receptor fusion molecule of the corresponding activity becomes effective and a self-amplifying release of the fusion proteins or the signaling molecules from the substrate is initiated.

In the release of a receptor fusion protein as a result of binding to a signaling molecule that is secreted by microorganisms the corresponding enzymatic activity becomes effective. After release from the surface of the substrate the released receptor fusion protein acts enzymatically onto still bonded receptor molecules or signaling molecules and detaches them from said surface. An individual or a few signaling molecules cause release of a self-amplifying cascade of releases from the surface of the substrate. In this way, a detection of marginal processes by use of quorum sensing is possible.

Advantageously, the additional protein, according to the embodiment of claim 14, has a proteolytic or nucleolytic or lipolytic function, for example, a protease or restriction endonuclease or a lipase.

For generating a signal cascade, a fusion of the receptors with proteins or peptides that have a specific nuclease or protease activity, e.g. a restriction endonuclease or a TEV protease, is realized. At the same time, binding of the receptor or the signaling molecule to the surface of the substrate by means of a linker is realized, which linker is comprised in the first case of a double-stranded DNA with the recognition sequence for the respective restriction endonuclease or in the second case of a peptide linker with recognition sequence for the respective protease. After detachment of the fusion proteins, comprised of receptor and endonuclease or protease, the fusion protein acts on the substrate-bonded linker sequences and amplifies thus the signal. The fusion is realized preferably at the DNA level by means of suitable recombinant DNA techniques.

According to the embodiment of claim 15, signaling molecules are advantageously bonded to portions of a surface of a substrate and receptors provided with fluorescence markers are attached thereto.

According to the embodiment of claim 16, the fluorescence markers can be of the same type or of different types in this connection. Advantageously, several fluorescence markers of different fluorescence can be assayed on the surface.

Preferably, according to the embodiment of claim 17, either fluorescence proteins or quantum dots can be used.

Accordingly, different microorganisms, for example, in mixed cultures can be assayed. Binding of the alternative fusion constructs to the optical sensor is done by means of the respective matching signaling molecule. Depending on the respective sensor principle, the immobilization of the specific autoinducers or receptors is locally defined or undefined.

In this way, simultaneous assaying of quorum sensing activities of different microorganisms on one in the same sensor platform is possible.

According to the embodiment of claim 18, the signaling molecule is an autoinducer-1 (AI-1), an autoinducer-2 (AI-2), a further signaling molecule or at least one combination thereof.

The receptor according to the embodiment of claim 19 is either a LuxP molecule or a gene-technologically generated derivative.

In addition to modifications that are carried out at the receptor, the respective signaling molecule can be modified in order to optimize the assay depending on the respective sensor. For example, for mass-selective sensors the mass of the signaling molecule can be preferably increased in that molecules are chemically coupled to the signaling molecule. For optical sensors, corresponding dyes can be coupled. In case of charge detectors, charges can be generated or modified.

Also, the binding affinity between receptor and signaling molecule can be modified by modifications at the signaling molecule. In this way, groups interacting with the receptor can be modified. Also, in case of use of AI-2 as ligand it can be expedient to integrate, instead of boron, a different element into the signaling molecule in order to affect in this way the binding properties or the steric orientation of the receptor.

The substrate according to the embodiment of claim 21 is advantageously a piezoelectric transducer. In a piezoelectric transducer the piezoelectric effect is utilized for conversion of force or pressure into an electric voltage. The occurring charges are proportional to the force that is acting. The charges can be detected as voltage so that the substrate itself can be a mass sensor. Changes of the acting forces cause directly corresponding charge changes and the resulting detectable voltage changes. Piezoelectric transducers are suitable advantageously for dynamic force measurement even at very high frequencies so that it is also possible to measure fast force changes by means of the equivalent voltage changes. With increasing resonance frequency of the piezoelectric transducer the sensitivity relative to mass changes increases. In this way, a very sensitive biosensor for detection of microorganisms is realized in the form of a micro scale.

Depending on the utilized sensor principle, the quorum sensing components used for functionalization can be selected in a targeted fashion. For the sensor system by means of a micro scale of a piezoelectric transducer it is, for example, expedient to immobilize the autoinducer having a significantly reduced mass relative to the receptor on the sensor surface and to detect the mass change that is caused by the detachment of the receptor from the signaling molecules immobilized on the substrate surface.

The substrate according to the embodiment of claim 22 is an electrode of a controllable semiconductor component or of a capacitors so that a charge sensing device is realized, respectively.

On the one hand, this is a controllable semiconductor element, advantageously in the form of a field effect transistor wherein the charge carrier transport is realized only by means of majority carriers. Binding, redistribution and detachment of charges at the substrate surface cause a measurable change of the conductive channel in the field effect transistor.

On the other hand, this is a capacitor wherein binding and immobilization of the receptor or of the autoinducer causes a change of electric energy in the electric field between the electrode as a dipole of a capacitor.

The substrate, according to the embodiment of claim 23, is a photodetector and/or an optoelectric component. Occurring fluorescence changes can be measured directly by means of fluorescence spectrometer as a photodetector. By means of coupling an optoelectric component the effect of fluorescence can be triggered or amplified.

The substrate according to the embodiment of claim 24 is a surface plasmon resonance spectrometer as an electron density sensor. An advantage is the high sensitivity of the plasmon resonance frequency relative to changes in the integral electron density of the immobilized protein coating as well as the excellent integration possibility into a microfluidic system.

On the substrate according to the embodiment of claim 25 a gold electrode array as an electrochemical sensor is present so that changes in the protein coating can be immediately detected by means of current-voltage characteristics in a suitably selected reference medium.

According to the embodiment of claim 26, the substrate is a component of a magnetic component. The substrate is used as a switching, amplifying or storing component. In this connection, it is advantageously a component of a logic circuit. A further advantage resides in that such a component can be switched from the exterior by means of logic operations. In this way, for example, the initial state can be reached again.

The substrate according to the embodiment of claim 27 is a calorimeter for enzymatic reactions that detects the temperature changes caused by the enzymatic reaction. The heat energy is correlated to the number of bonded enzymatically active receptor molecules.

With the aid of the following Figures and examples the invention will be explained in more detail. It is shown in:

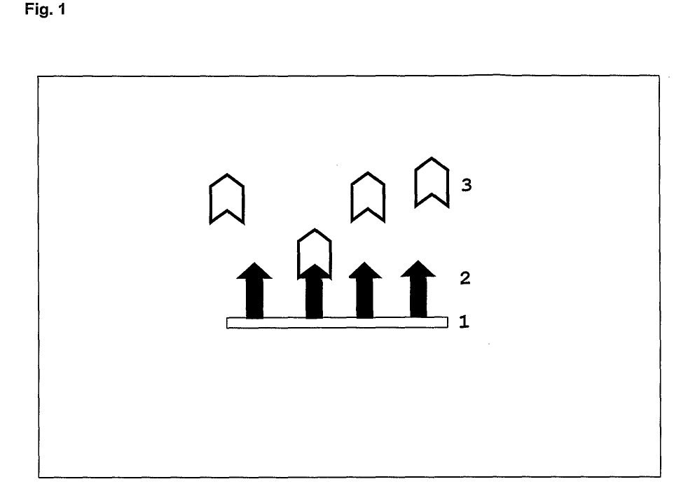

FIG. 1: immobilization of the receptor on the substrate surface;

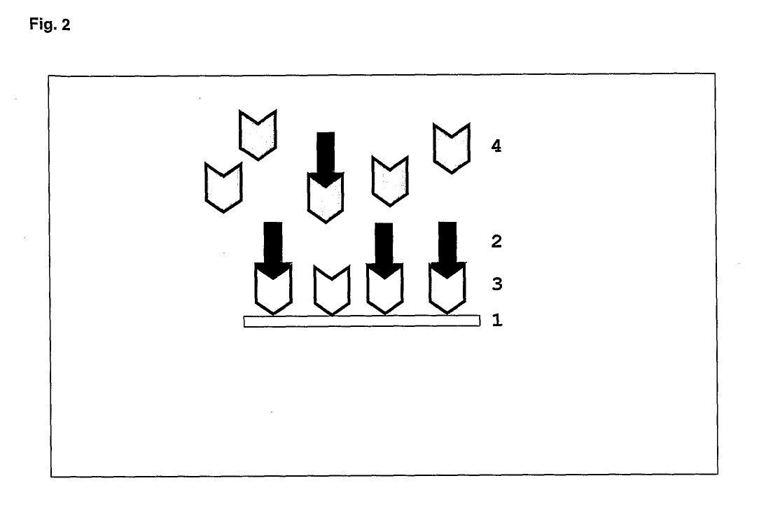

FIG. 2: immobilization of the autoinducer on the substrate surface.

### FIRST EMBODIMENT

FIG. 1: The receptor or corresponding derivatives (2) are immobilized chemically on a surface of the substrate (1). Autoinducer molecules (3) contained in the culture medium bind to the receptors or their derivatives. The thus resulting physical or physicochemical changes are detected by sensors.

For producing a biosensor from a LuxP protein as receptor (2) on the surface of the substrate (1), the gene that is coding for the LuxP protein is PCR-amplified with appropriate primers, for example, from genomic DNA. The PCR fragment is purified and cloned into an expression vector, for example, pET-23 or novagen, for E. coli and the sequence of the gene is verified by means of sequence analysis. By cloning into the vector, the receptor is provided at the C-terminal end with a histidine tag. After expression of the gene the receptor protein is purified by means of affinity chromatography by Ni-NTA columns (Qiagen).

The thus obtained LuxP protein is bonded by a suitable linker to the surface of the substrate (1) that connects covalently the inorganic surface of the substrate (1) with the respective biomolecule.

Binding of the receptor proteins is realized preferably by known methods for binding proteins to substrate surfaces. Suitable binding possibilities are, for example:
in case of a substrate surface made from gold, the linker is bonded by a thiol to the gold surface, for example, 11-mercapto undecane acid, and forms by a carboxylic group that is activated by means of N-(3-dimethylaminopropyl)-N′-ethylcarbodiimide (EDC) and N-hydroxy sulfosuccinimide (NHS) an amide bond with primary amines of the protein, or
in case of substrates with metal oxide surfaces, the protein is bonded by an silane presenting an epoxide-group, for example, 3-glycidyloxypropyl trimethoxysilane, to the substrate surface that forms with primary amines of the protein secondary amine bonds.

### SECOND EMBODIMENT

FIG. 2: autoinducers or corresponding derivatives (3) are immobilized chemically on the surface of the substrate (1). Corresponding receptor molecules or their derivatives (2) are bonded by the autoinducers to this surface. Autoinducer molecules (4) contained in the culture medium compete in regard to binding to the receptors or their derivatives and cause their detachment. The thus resulting physical or physicochemical changes are detected by sensors.

For producing a biosensor from a signaling molecule or its derivatives (3) immobilized on the surface of the substrate (1) and a receptor or its derivatives (2) coupled thereto, the autoinducer is chemically synthesized and bonded to the surface by means of a suitable linker. The linker links the inorganic surface covalently to the corresponding autoinducer.

The autoinducer can be bonded by a homobifunctional crosslinker, for example, 1,4-butanediol diglycidyl ether, to the substrate that forms ether bonds with hydroxyl groups of the autoinducer. Binding to the substrate surface is realized by bonding of crosslinker to layers of the substrate that present amino groups, cysteine monolayer in the case of gold surfaces or aminosilanes or amino terpolymers in case of oxide surfaces. The autoinducer molecule can be bonded also by means of EDC/NHS activated 11-mercapto undecane acid to the gold surface of the substrates.

The gene that codes for LuxP is amplified by PCR by means of appropriate primers, for example, from genomic DNA. The PCR fragment is purified and cloned into an expression vector, for example, pET-23 or novagen, for E. coli, and the sequence of the gene is verified by sequence analysis. By cloning into the vector, the receptor is provided at the C-terminal end with a histidine tag. After expression of the gene the receptor protein is purified by means of affinity chromatography by Ni-NTA columns (Qiagen).

The thus obtained protein (2) is bonded by the autoinducer (3) to the surface of the substrate (1). Autoinducer molecules (4) present in the culture medium compete in binding to the receptors and cause their detachment, leading to physical or physicochemical changes. These changes are detected by sensors.

The substrate (1) of embodiments is advantageously at the same time a sensor.

In a first embodiment the substrate (1) is a piezoelectric transducer. The transducer itself is a transducer element of piezoceramic material provided with electrodes. The transducer element itself is a plate-shaped or film-like body. The electrodes of the transducer element are, as is known in the art, connected by means of an amplifier with high-resistance input to a voltmeter that is, at the same time, a component of a data processing device. In this way, the attachment and detachment of receptors on the surface of the transducer element can be measured as measured values by means of the correlated mass changes on this surface. The transducer element functions as a micro scale. Advantageously, these measured values are saved in the data processing device so that an evaluation in accordance with various criteria can be done easily.

In a second embodiments, the substrate (1) is the gate as an electrode of a field effect transistor. By attachment and detachment of receptors at the gate equivalent charge changes occur so that also the channel resistance between source and drain changes equivalently. This resistance change is measured as current or voltage change and acquired by a data processing unit attached thereto, saved, and displayed.

In a third embodiment the substrate (1) is at least one of the electrodes of a capacitor. The culture medium acts at the same time as a dielectric between the electrodes of the capacitor. The attachment and detachment of receptors causes charge changes and/or charge displacements at the electrodes that can be measured by applying alternating current. The capacitor acts thus as an alternating current resistance. The capacitor is advantageously connected to a data processing unit for this purpose. The capacitor itself can be configured as a plate capacitor, a spherical capacitor or a cylindrical capacitor.

In a fourth embodiment the substrate (1) is a photodetector and/or an optoelectric component. In this connection, the photodetector is advantageously an optoelectronic semiconductor component wherein free charge carriers are generated by absorption of light. Such photodetectors are in particular a photoconductive cell, a photo diode, a photo transistor, and photo thyristor. The optoelectric component is in the form of a known luminescence diode that can also be in the form of a laser diode. The photodetector and the optoelectric component are connected to a data processing unit for controlling and acquisition. In a first variant the photodetector and the optoelectric component are arranged at a spacing from one another wherein the culture medium is arranged between them. In a second variant, the photodetector and the optoelectric component are arranged adjacent to one another in a plane. At a spacing thereto, there is a component that predominantly reflects light. The culture medium in this connection is located between the photodetector and the optoelectric component, on the one hand, and the component, on the other hand.

In a fifth embodiment the substrate 1 is a surface plasmon resonance spectrometer as an electron density sensor that is characterized by a high sensitivity of the plasmon resonance frequency relative to changes in the integral electron density of the immobilized protein coating as well as by excellent integration possibility into a microfluidic system.

In a sixth embodiment, on the substrate 1 there is provided a gold electrode array as an electrochemical sensor so that changes in the protein coating can be detected immediately by current-voltage characteristics in a suitably selected reference medium.

In a seventh embodiment the substrate 1 is a calorimeter for enzymatic reactions with which temperature changes caused by the enzymatic reactions are detected. The calorimeter is connected to a data processing unit.

The data processing units of the embodiments are advantageously known computers.

### LIST OF REFERENCE NUMERALS

1 surface of the substrate
2 receptor
3 signaling molecule
4 signaling molecule that is released by microorganisms in the process of quorum sensing and therefore is present in the medium.

What is claimed is:

1. Method for detection of microorganisms and/or their activity with biosensors, comprising the steps of immobilizing chemically, physically, or biologically on a surface of a substrate over portions thereof at least one ligand for binding a receptor or at least one receptor for a ligand or at least one ligand for binding a receptor and at least one receptor for either the one or a further ligand and measuring physical or physicochemical changes, caused at receptors by binding of ligands that are emitted by microorganisms in the process of quorum sensing.

2. Method according to claim 1, wherein on the surface of the substrate over portions thereof at least one ligand is bonded, receptors are bonded to ligands, receptors bind ligands that are emitted by microorganisms in the process of quorum sensing and/or that are already present in the medium, wherein receptors are detached by ligands of the medium and wherein the detachment is detected measuring technologically by the caused changes of the charge, the mass, the conformation, the optical properties, the magnetic properties or the enzymatic activity.

3. Method according to claim 1 wherein the ligand is a signaling molecule.

4. Method according to claim 3, wherein on the surface of the substrate over portions thereof at least one receptor is bonded, signaling molecules released by microorganisms in the process of quorum sensing and/or already present in the medium are bonded to receptors, wherein a physicochemical change is realized so that measuring technologically the thus caused changes of the charge, the mass, the conformation, the optical properties, the magnetic properties, the charge distribution or the enzymatic activity are detected.

5. Method according to claim 1, comprising the step of calibrating the biosensor by attachment of particles on the ligands or receptors.

6. Method according to claim 3, comprising the step of regenerating the surface of the substrate with signaling molecules and receptors bonded thereto by a repeated coating with the receptor.

7. Method according to claim 1, comprising the step of modifying, for generating fusion proteins by means of recombinant techniques, amino acid sequences of receptors such that a change of charge, mass, light, conformation or enzymatic reaction takes place and/or that a change of charge, mass, light, conformation or enzymatic reaction is done chemically or physically.

8. Method according to claim 7, wherein receptors are modified by fusion of charged peptides to fusion proteins with changed net charge.

9. Method according to claim 8, wherein the peptides comprise preferably arginine, histidine, or lysine for a positive net charge or that the peptides comprise aspartic acid or glutamic acid for a negative net charge.

10. Method according to claim 7, wherein receptors are modified by fusion of proteins or peptides such that the mass of the fusion protein is significantly increased.

11. Method according to claim 7, wherein receptors are modified by fusion of proteins or peptides that exhibit autofluorescence or that cause enzymatically by adding suitable substrate molecules a luminous reaction.

12. Method according to claim 3, comprising the step of modifying receptors in their amino acid sequences such that the affinity between signaling molecule and receptor is increased or decreased.

13. Method according to claim 3, by recombinant techniques an additional protein is added to receptors so that fusion proteins are generated and that either the fusion proteins or the signaling molecules are bonded to the surface of the substrate by amino acid or nucleic acid sequences or lipids that are sensitive relative to the enzymatic activity of the fusion proteins so that upon release of a receptor fusion molecule the corresponding activity is activated and a self-amplifying release of the fusion proteins or signaling molecules from the substrate is initiated.

14. Method according to claim 13, wherein the additional protein has a proteolytic, a nucleolytic or a lipolytic function.

15. Method according to claim 3, on the surface of the substrate over portions thereof at least one signaling molecule is bonded and that receptors provided with fluorescence markers are bonded to signaling molecules.

16. Method according to claim 15, wherein the fluorescence markers are of the same type or of different types.

17. Method according to claim 15, wherein the fluorescence markers are fluorescence proteins or quantum dots.

18. Method according to claim 3, wherein the signaling molecule is an autoinducer-1 (AI-1) and/or an autoinducer-2 (AI-2) and/or a further signaling molecules or at least a combination thereof.

19. Method according to claim 1, wherein the receptor is either a LuxP molecule or a gene-technologically produced derivative.

20. Device for detection of microorganisms and/or their activity as biosensor, comprising a substrate, wherein on a surface of the substrate over portions thereof at least one ligand for binding a receptor or at least one receptor for a ligand or at least one ligand for binding a receptor and at least one receptor for either the one or a further ligand is immobilized chemically, physically or biologically, wherein measuring means are provided that measure physical or physicochemical changes caused by bonds between the ligands that are released by microorganisms in the process of quorum sensing and the receptors.

21. Device according to claim 20, wherein the substrate is a piezoelectric transducer.

22. Device according to claim 20, wherein the substrate is an electrode of a controllable semiconductor component or of a capacitor.

23. Device according to claim 20, wherein the substrate is at least one photodetector and/or at least one optoelectric component.

24. Device according to claim 20, wherein the substrate is a surface plasmon resonance spectrometer as an electron density sensor.

25. Device according to claim 20, wherein on the substrate a gold electrode array as an electrochemical sensor is arranged.

26. Device according to claim 20, wherein the substrate is a component of a magnetic component.

27. Device according to claim 20, wherein the substrate is a calorimeter for enzymatic reactions that detects temperature changes generated by the enzymatic reactions.
